(19) United States
(12) Patent Application Publication
(10) Pub. No.: US 2007/0055138 A1
(43) Pub. Date: Mar. 8, 2007

(54) Accelerated whole body imaging with spatially non-selective radio frequency pulses

(76) Inventor: Robert R. Edelman, Highland Park, IL (US)

Correspondence Address: Jon P. Christensen;Welsh & Katz, Ltd., 22nd Floor, 120 South Riverside Plaza, Chicago, IL, 60606, US

(21) Appl. No.: 11/208,845

(22) Filed: Aug. 22, 2005

(51) Int. Cl. A61B 5/05

(52) U.S. Cl. 600/411

(57) ABSTRACT

A method and apparatus are provided for forming a magnetic resonance image of a human. The method includes the steps of applying a plurality of relatively constant spatially non-selective radio frequency pulses to an imaging volume of the human, applying a plurality of combinations of magnitude of phase-encoding gradients in slice-selective and in-plane directions to the imaging volume of the human, wherein the plurality of combinations is adapted to undersample the imaging volume in k-space, detecting magnetic resonance imaging data from the imaging volume using a plurality of receiver coils and forming the magnetic resonance image of the imaging volume.

### FIELD OF THE INVENTION

The field of the invention relates to computed tomography and more particularly to magnetic resonance imaging.

### BACKGROUND OF THE INVENTION

Arterial diseases and injuries are common and have severe consequences including amputation or death. Atherosclerosis, in fact, is a major problem in the aged population, particularly in the developed countries.

Atherosclerosis of the lower extremities (often, otherwise, referred to as peripheral vascular disease) is a common disorder that increases with age, ultimately affecting more than 20% of those people over the age of 75. Lesions resulting from atherosclerosis are often characterized by diffuse and multi focal arterial stenosis and occlusion.

Peripheral vascular disease often manifests itself as an intermittent insufficiency or claudication of blood flow in calf, thigh or buttocks. The symptoms of claudication often result from an inability of the body to increase blood flow during exercise.

In more extreme cases of peripheral vascular disease, blood flow of even a resting patient may be insufficient to meet basal metabolic needs of the extremities. Symptoms of blood flow insufficiency in these areas may include pain in the forefoot or toes or, in extreme cases, non-healing ulcers or gangrene in the affected limb.

One of the most effective means of diagnosing and treating atherosclerosis is based upon the use of magnetic resonance angiography (MRA) to create images of portions of the vascular system. As is well known, MRA is a form of magnetic resonance imaging (MRI) which is especially sensitive to the velocity of moving blood. More specifically, MRA generates images by relying upon an enhanced sensitivity to a magnitude and phase of a signal generated by moving spins present within flowing blood.

MRA, in turn, can be divided into three types of categories: 1) time of flight (TOF) or inflow angiography; 2) phase contrast (PC) angiography (related to the phase shift of the flowing proton spins) and 3) dynamic gadolinium enhanced (DGE) MRA. While the three types of MRA are effective, they all suffer from a number of deficiencies.

The predominant deficiency of all three types of existing MRA techniques relates to speed of data collection. For example, patient motion is known to significantly degrade image quality of TOF MRA. To avoid image degradation, a patient undergoing DGE MRA is typically required to hold his breath during data collection. PC MRA relies upon the use of long time-to-echo (TE) intervals for signal sampling that result in other T2 effects that tend to degrade image quality. Because of the importance of MRA, a need exists for MRA methods that are less reliant upon time or upon movement of the patient.

### SUMMARY

A method and apparatus are provided for forming a magnetic resonance image of a human. The method includes the steps of applying a plurality of relatively constant spatially non-selective radio frequency pulses to an imaging volume of the human, applying a plurality of combinations of magnitude of phase-encoding gradients in slice-selective and in-plane directions to the imaging volume of the human, wherein the plurality of combinations is adapted to undersample the imaging volume in k-space, detecting magnetic resonance imaging data from the imaging volume using a plurality of receiver coils and forming the magnetic resonance image of the imaging volume.

### BRIEF DESCRIPTION OF THE DRAWINGS

FIG. 1 is a block diagram of a magnetic imaging system in accordance with an illustrated embodiment of the invention;

FIG. 2 depicts a pulse sequence that may be used by the system of FIG. 1; and

FIG. 3 depicts projection signals of the body of FIG. 1 along various axis.

### DETAILED DESCRIPTION OF AN ILLUSTRATED EMBODIMENT

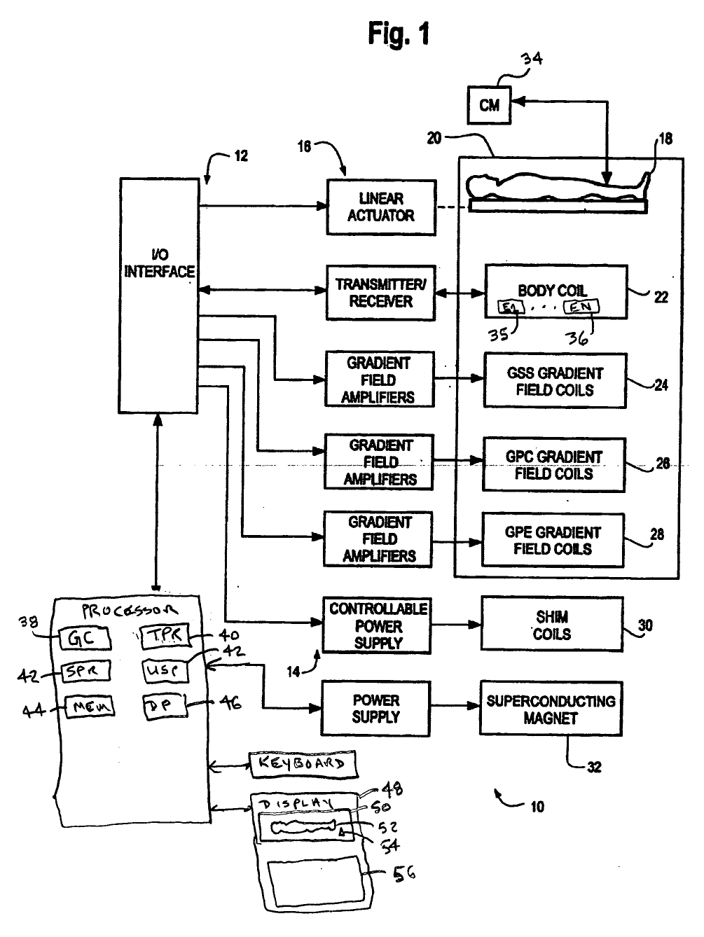

FIG. 1 is a block diagram of a magnetic resonance imaging system 10 under an illustrated embodiment of the invention. While the system 10 is amenable to use in a number of different contexts, the methods described below differ significantly from those of prior systems. For instance, compared to the prior patents (U.S. Pat. Nos. 6,728,569 and 6,901,282 invented by the inventor of the instant invention) the system 10 greatly accelerates data acquisition and incorporates the ability to depict dynamic processes such as the passage of a contrast agent through various portions of the vascular system. Unlike prior systems, the system 10 permits the simultaneous acquisition of a high resolution 3-D data and also data for time-resolved images.

The methods described below represents a significant improvement over the applicant's prior patents for other reasons. For example, the applicant's prior patents apply to imaging techniques that can be acquired at multiple stations to encompass entire imaging volumes through the use of non-selective RF excitations. There is a significant potential limitation, however. Even using spatially non-selective RF excitation and very short repetition times (TR), breath-hold times can be excessive if an entire volume is encompassed with thin slices.

Other patents, in the same technology, have also failed to address this problem for various reasons. For example, the VIPR technique of Mistretta et al. (U.S. Pat. No. 6,487,435) requires the use of projection reconstruction methods and excludes the use of Cartesian methods, unlike the proposed method. Moreover, the method of Mistretta violates the Nyquist condition for sampling a peripheral region of k-space, unlike the proposed method which satisfies the Nyquist condition and is thus free from undersampling artifacts. The Mistretta method also does not incorporate rapid MRI data acquisitions for the purpose of time-resolved imaging.

The method described by Goldfarb, J W et al., in “Simultaneous Magnetic Resonance Gadolinium-Enhanced 2D Perfusion and 3D Angiographic Imaging” (Magn. Reson.

Imaging, 585-591, Jul. 21, 2003) fails to take advantage of the acceleration inherent in the use of high acceleration factors (two-fold or greater) for k-space undersampling and limits the time-resolved acquisition to a single-slice, 2D perfusion imaging sequence which could not be used to image blood vessels, for instance. The method of 0. Heid (Proc. Intl. Soc. Mag. Reson. Med., 8 (2000) 1784) uses a nonselective RF excitation but fails to take advantage of the acceleration inherent in the use of high acceleration factors (two-fold or greater) for k-space undersampling nor does it allow time-resolved imaging. The method of J P Finn et al. (Radiology 2002; 224:896-904) allows time-resolved angiographic imaging but only with low spatial resolution and fails to take advantage of the acceleration inherent in the use of high acceleration factors (two-fold or greater) for k-space undersampling. Finally, these prior method are only useful for the specific application of blood vessel imaging (MR angiography) whereas the proposed invention is more generally applicable for contrast-enhanced imaging of any organ system.

Under a first embodiment, data acquisition may be accelerated by combining k-space undersampling, a high sampling bandwidth, receiver coils with two or more elements and spatially non-selective RF excitation, so as to reduce scan times by a factor of two or more. The use of substantial k-space undersampling or receiver coils with multiple elements has not been previously recognized as being compatible with the concepts of non-selective RF excitation.

By this combined approach, data can be acquired for each entire imaging volume within a single breathholding period, even when thin slices (e.g., 3 mm or thinner) and large numbers of 3D partitions (96 or more) are acquired. The method can be used to acquire data for a single imaging volume, even if scout imaging is not performed. Alternatively, data can be acquired for multiple imaging volumes at different locations, even if scout imaging is not performed. Moreover, the method is not limited to the acquisition of angiogram-like pictures, but can be used more generally for many clinical applications such as liver or breast imaging for detection of tumors. Like the patented methods of the inventor above, the proposed method does not require the use of scout imaging for positioning of the 3D acquisition volume, although use of scout imaging is not precluded.

In general, MRI is routinely used for the diagnosis of a wide variety of diseases. Scout images are obtained prior to the acquisition of MR images in order to guide proper positioning of high-resolution MRI acquisitions. As MR systems are developed that make it easier to acquire images of the entire body, it would be desirable to simplify and accelerate the scout imaging process, as well as reduce the amount of manual intervention by the operator.

Under a second embodiment, the system 10 may acquire up to three orthogonal full-thickness projections of the body, thereby showing the entire extent of tissue in as many as three dimensions. The extent of the tissue of each of the three dimensions is determined by an automated edge-detection processing that detects the threshold between soft tissue and air outside the body. The projections can be acquired so rapidly as to be nearly real-time and do not add measurably to the duration of the MRI study. If desired, the MRI table can be translated to bring a different portion of the body into the field of view of the magnet and RF coil used for signal reception, and the projection scout process repeated until the entire body had been scanned. The method can be used to automatically determine when the last portion of the body has been encompassed and the scout process discontinued.

The process may be particularly helpful to guide the rapid acquisition of a series of 3-D data sets spanning the entire thickness, breadth, and length of the body. The data sets can be combined and then a surface or volume rendered so as to produce a single “homunculus” displaying the surface and/or interior structures of the entire body. The ability to rapidly create a “homunculus” is beneficial for the efficient and accurate positioning of additional data acquisitions using a graphical user interface (GUI), as well as for providing a rapid evaluation of normal and abnormal anatomy throughout the body.

As shown in FIG. 1, the system 10 for collecting MR images of a patient 18 may include three subsystems 12, 14, 16. A patient movement subsystem 16 may be used to control the movement of a patient transport table within a scanning zone 20 of the system 10. A signal processing subsystem 14 may provide the magnetic fields and control transmission and detection of radio frequency (RF) signals from resonant atoms within the patient 18. A control subsystem 12 may provide programming and control of the first and second subsystems 14, 16. The first and second subsystems 14, 16 may be conventional.

A body coil 22 may be used for the transmission of RF pulses and to detect resonant signals. The body coil 22 may be provided in the form of a phased array coil with no fewer than two and as many as eight or more elements 35, 36.

First, second and third gradient field coils 24, 26, 28 may be used to create and control gradient magnetic fields within the body coil 22. A superconducting magnet 32 and shim coils 30 may be used to provide a static magnetic field within the scanning zone 20.

In order to prepare the patient 18 for imaging, a contrast agent (e.g., gadolinium-chelate) 34 may be injected into the patent 18. The contrast agent 34 may be administered using any appropriate method (e.g., hypodermic needle).

To collect image data through the thickness of the body, a spatially non-selective RF pulse may be applied through the body coil 22 without the necessity for any, or only a relatively low level, slice selective gradient Gss that would otherwise be applied at the same time as the RF pulse. Because of the relatively constant frequency of the spatially non-selective RF pulse and the absence of phase-encoding gradients, the spatially non-selective RF pulse need only be a fraction of the length of a spatially selective RF pulse. Also, because of the short duration of the spatially non-selective RF pulse, the minimum repetition time is much shorter. Repetition rates of less than 3 milliseconds (ms), in fact, are possible using the spatially non-selective RF pulse.

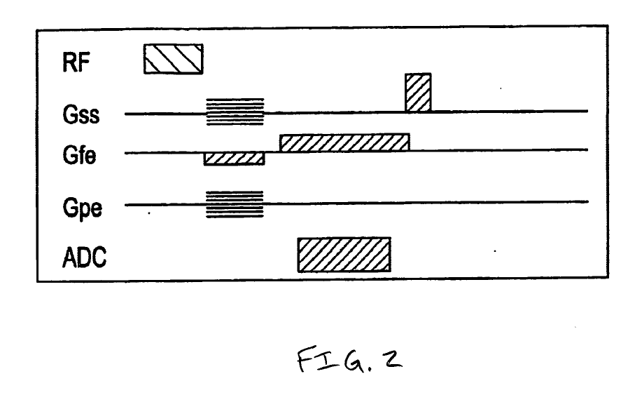

FIG. 2 depicts a 3D gradient-echo pulse sequence using the spatially non-selective RF pulse. In other embodiments, gradient echo, steady-state free precession, spin-echo, fast spin-echo, echo planar or other pulse sequences may be used for data acquisition.

As shown in FIG. 2, the RF pulse may remain relatively constant among pulse sequences, as does the frequency encoding gradient Gfe and the timing of data collection through the analog-to-digital converter (ADC). The absence of any slice selection gradient during the RF pulse should be specifically noted in FIG. 2. The absence of any slice selection gradient during the RF pulse allows the RF pulse to be spatially non-selective in its effect on resonant atoms.

In order to collect data based upon each spatially non-selective RF pulse of FIG. 2, the phase-encoding gradient Gss, in the slice direction and the phase-encoding gradient Gpe in the in-plane direction may be varied by a gradient controller 38 in some predetermined manner. As used herein, varying the phase-encoding gradients Gss, Gpe means applying a number of phase-encoded gradient combinations among pulse sequences (after the RF pulse has ended) in the slice selective and in-plane directions while collecting data for each combination under conditions of a constant frequency-encoding gradient Gfe and constant three-dimensional spatially non-selective frequency pulses RF among the pulse sequences.

For example, the full-scale range of the phase-encoding gradients in the slice and also the in-plane directions may each be divided up into a number of incremental steps (e.g., 64-256). Data may be collected by selecting a value for the first phase-encoding gradient while varying a value of the second phase-encoding gradient. After collecting data over a range of values for the second phase-encoding gradient, a new value may be selected for the first phase-encoding gradient and the process may be repeated until a full complement of data has been collected. A full complement of data may mean collecting data for each combination of phase-encoded gradients within an imaging area.

As a further, more detailed example, a lowest relative value may be chosen for the first phase-encoding (e.g., the slice selective) gradient. Next a lowest relative value of the second phase-encoding (e.g., the in-plane) gradient may be selected and a first set of data may be collected using these two phase-encoding values via the use of the sequence of FIG. 2. Following collection of the first set of data, the phase-encoding value of the second phase-encoding gradient may be incremented and a second set of data may be collected.

The process of incrementing the second phase-encoding gradient value (and collecting data sets) may be repeated until a maximum gradient value is achieved for the second phase-encoding gradient. Once the maximum value is achieved for the second phase-encoding gradient, the first phase-encoding gradient may be incremented and the process may be repeated. The process may be repeated by as many steps that it takes to increment the first phase-encoding gradient from a minimum value to a maximum value.

In order to further enhance processing efficiency, the system 10 may function to identify the presence, location and thickness of any body portions of the patient 18 within each slice. Once identified, a thickness processor 40 of the system 10 may function to limit data collection to the location and to the thickness of any identified body portions.

As a first step, the system 10 may perform a coarse scan of each slice. A slice processor 42 may then determine whether the slice passes through any part of the body of the patient 18. The slice processor 42 may make this determination by comparing a resonance value of each voxel of the slice with a threshold value. If the resonance values of each voxel of the slice exceed the threshold value (indicating that the slice does not pass through any body portions), then the system 10 may discard the slice.

If it is determined that some part of the slice passes through the patient 18, then the system 10 may group the voxels of the body portion(s) and identify an outer boundary of the body portion(s) within the slice. As a first step, a thickness processor 40 may determine a center of the body part (i.e., the center of each significant group of voxels that do not exceed the threshold value). This may be performed using a simple grouping and weighting algorithm.

The thickness processor 40 may then calculate the thickness of each body portion based upon average resonance values of the voxels within the body portions of the slice. To determine an average value, the processor 40 begins by selecting a value at a center of the body portion as a first average value and averaging outwards. As each new voxel value is examined, it is compared with the average. If it is within a threshold value of the average, it may be incorporated into the average. If it is not, then the voxel location and value may be segregated as a potential boundary area of the body.

A line tracing routine within the slice processor 42 may attempt to connect boundary voxel locations that exceed the threshold (where each boundary voxel lies adjacent other voxel locations that do not exceed the threshold). If the line tracing routine is able to successfully trace a continuous line around the center of the slice, then the line is assumed to define the boundary of the portion of the body 18 within the slice. The diameter of the traced boundary line defines the thickness of the body portion within the slice.

In addition to discarding slices outside the body and in addition to limiting imaging processing to portions within the body, the coarse images may also be used to reduce scanning times by automatically choosing minimum and maximum phase-encoding gradients in the slice-selective and in-plane directions. In this regard, the slice processor 42 may identify a set of minimum and maximum phase-encoding gradients in the slice-selective and also in the in-plane directions that identify voxels on the periphery of the body 18.

An incremental step size in the slice-selective and in-plane direction may then be automatically determined by the gradient processor 38. For example, for a particular slice-selective gradient value, the gradient processor 38 may determine in-plane gradient values that correspond to voxel positions on opposing sides of the body 18. The gradient processor 38 may determine a distance between the opposing voxels and divide by the desired slice thickness (entered by an operator of the system 10) in that direction to automatically obtain the size of the incremental in-plane gradient values in that direction. Alternatively, the operator could enter the number of slices in that direction and the gradient processor 38 may automatically determine a slice thickness. For a particular, in-plane gradient value, the gradient processor 38 may follow the same process to determine a set of incremental slice-selective gradient values.

Once a set of incremental values have been selected for a particular slice, the values may be transferred to a undersampling processor 42. K-space undersampling may be accomplished by the undersampling processor 42 by use of partial Fourier or partial k-space sampling along either or both the slice-selection and/or phase-encoding directions. Where the undersampling processor 42 uses partial k-space sampling, the incremental values may be adjusted by an undersampling factor to further increase scanning speed (decrease data collection time). For example, for an undersampling factor of 2.0, the undersampling processor 42 may double the incremental gradient values in either the slice-selective or the inplane directions to reduce the scanning time by one-half.

Reconstruction of the undersampled data into a full data set may be accomplished conventionally using a parallel reconstruction technique. Parallel reconstruction techniques (e.g., SENSE, ASSET, GRAPPA, etc.) may be applied in one or multiple directions. The data may also be processed using maximum intensity projections, multi-planar reconstructions, surface or volume renderings. In addition, images acquired before administration of a contrast agent 34 may be subtracted from images acquired after contrast administration so as to produce different which the signal intensity of unenhanced tissues is reduced.

Using the phase-arrayed coil 22 and process described above, data may be collected within each x-y plane along a length of the body 18. As a magnitude of each data element is collected, the data element may be stored in a memory 44 using Cartesian coordinates. While the examples below will be based upon a rectilinear coordinate system, spiral or radial k-space sampling patterns may alternately be used to acquire and store the data.

In addition to saving each data element and the 3-D source coordinates of the data element, the system 10 may also store the 3-D coordinates of the periphery of the body 18. Alternatively, the system 10 could collect 3-D coordinates of the periphery of the body 18 without collecting any further data. Using the 3-D coordinates of the periphery of the body 18, a display processor 46 may provide a GUI 50 over a portion of the display 48. Within the GUI 50 may be a 3-D image (homunculus) 52 of an exterior of the body 18.

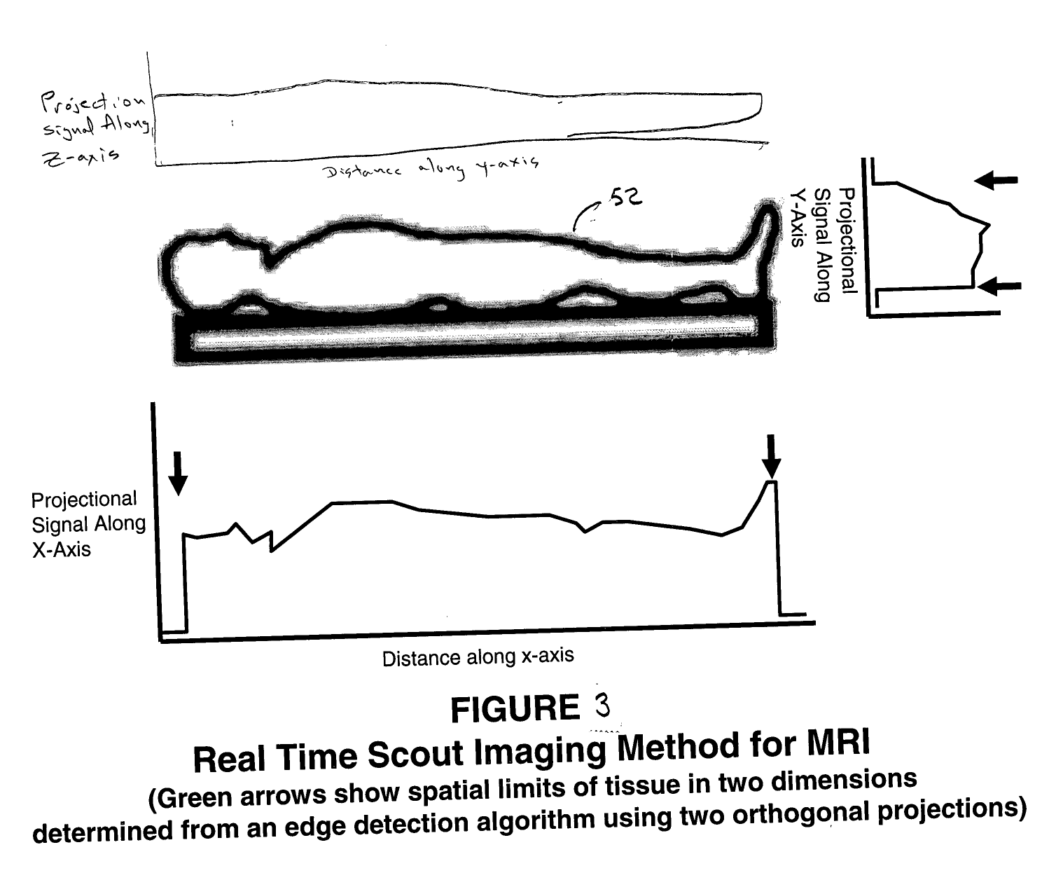

FIG. 3 shows projections along the x and y axis. Using a cursor 54, the operator of the system 10 may rotate the image 52 and select planes through the image 52, or projections (FIG. 3), for display of already collected data (in a slice viewing window 56) or to collect additional, more detailed data. If a plane is selected by the operator for existing data, then the display processor 46 simply retrieves the data from memory 44 and displays the image created by the data on the slice display 56.

If the operator selects a plane for collection of additional, more detailed data, then the operator may be presented with a screen for entry of scanning parameters (e.g., sampling bandwidth, in-plane encoding steps, samples in the frequency encoding direction, slice-selective encoding steps, slice thickness, sampling volume, etc.). In response, the slice processor 42 may generate a series of pulse sequences and collect the additional data as instructed and display the data within the display 56. To insure that the patient 18 has not moved, the slice processor 42 may generate one or more scout images and perform pattern matching to ensure that the new scout image matches the selected slice location.

In addition to generating additional detail regarding the slice or volume selected by the operator, the system 10 may also simultaneously collect time resolved images of a particular feature of the body 18. A time resolved image is a series of images that show the same anatomical feature over a period of time. In this case, there are two scanning processes occurring simultaneously.

In general, the first scanning process is a high spatial resolution (HSR) scan that uses a series of HSR pulse sequences to create high resolution images. The second scanning process is a time-resolved scan that uses a series of time-resolved pulse sequences that are interleaved with the HSR pulse sequences.

In general, the method has three key features. First, a non-selective RF excitation is used to excite an entire volume of tissue and used to create the HSR 3-D MRI. Second, a series of rapid 2-D or 3-D MRI data acquisitions are interleaved into the lengthier HSR MRI data acquisition so as to produce a series of time-resolved MR images. (Each time-resolved MRI is obtained in a small fraction of the duration of the HSR 3-D MRI.) Third, k-space is undersampled by a factor or two or more to reduce the scan time to a duration that allows all data to be obtained in a single breath-hold of the patent 18.

One important purpose of the non-selective RF excitation in this example is to ensure that the entire thickness of the body is imaged and no tissue is left out. This method obviates the need for careful positioning of the imaging volume, which would be the case with a selective RF excitation, and minimizes set-up time for the scan. Moreover, the non-selective RF excitation uses less RF power and allows for shorter repetition times (TR), which is particularly beneficial at higher magnetic field strengths such as 3 tesla. However, for HSR 3-D MRI using non-selective RF pulse, the scan time is much too long to observe the dynamic passage of contrast agent 34 through an organ system or vasculature. Moreover, the scan time may be too long to permit breath-holding, so that motion artifacts degrade the images particularly in regions like the chest and abdomen. Therefore, the method employs a combination of k-space undersampling by at least a factor of two and rapid acquisition of time-resolved images that is interleaved into the HSR 3-D MRI. Each time-resolved image, though of lower spatial resolution than an HSR 3-D MRI, has the additional advantage of being more resistant to motion artifact because it is acquired in a relatively shorter period of time.

With regard to the GUI 50, the operator of the system 10 can select the HSR data for viewing of an HSR image or the time-resolved data for a time sequence of images that show, for instance, the flow of the contrast medium 34 through the anatomical structure displayed in the time-resolved image. Alternatively, the operator may elect to have the GUI 50 show the time-resolved image superimposed over the HSR image.

A specific example of this method would involve undersampling of k-space and a self-calibrated parallel data reconstruction technique to reconstruct the data, a sampling bandwidth of 125 kHz and a phased-array receiver coil with eight elements. For the HSR 3-D MRI, the TR/TE is 2.5 msec/0.6 msec, flip angle of 25 degrees, 192 in-plane phase-encoding steps, 320 samples in the frequency-encoding direction, 128 phase-encoding steps in the slice-select direction interpolated to 256 slices (1 mm thick), a parallel acceleration factor of two, asymmetric sampling in the slice direction (for a further 25% reduction in scan time). For the time-resolved 3-D MRI, the TR/TE is 1.6 msec/0.5 msec, flip angle is 15 degrees, 128 in-plane phase-encoding steps, 192 samples in the frequency-encoding direction, 4 phase-encoding steps in the slice-select direction interpolated to 8 3-D slices (32 mm thick), parallel acceleration factor of four. With this example, the duration of the HSR acquisition is 23 seconds and the duration of the each time-resolved acquisition is 204 msec. The time-resolved acquisition is repeated at intervals of 2 seconds. In other examples, the flip angle of the RF excitation and/or RF pulse duration could be varied over the course of the acquisition so as to reduce the amount of power deposition.

In the example, more than one k-space undersampling method may be used concurrently in the example so as to achieve a total reduction of scan time of at least 2. In addition, the time-resolved data may have a different orientation than any of the three axis of the Cartesian coordinate system under which the HSR data is acquired. Further, data for a number of different time-resolved images may be collected concurrently with the HSR data. The number of time-resolved images may also have mutual angles that are different than multiples of 90 degrees.

The time-resolved data do not have to begin and end with collection with the HSR data. The time-resolved MRI may be continued for a period of time preceding and/or succeeding the HSR MRI. In addition, arbitrary time intervals may be used between the collection of time-resolved data.

In addition to the pulse sequences shown in FIG. 2, a magnetization preparation consisting of one or more RF pulses may be applied so as to alter tissue contrast. Chemical shift-based fat suppression methods may be used to reduce the contrast of certain tissue. Magnetization transfer may be used to further improve image quality as well as keyhole techniques and variations (e.g., TRICKS).

A specific embodiment of a method and apparatus for performing magnetic resonance imaging has been described for the purpose of illustrating the manner in which the invention is made and used. It should be understood that the implementation of other variations and modifications of the invention and its various aspects will be apparent to one skilled in the art, and that the invention is not limited by the specific embodiments described. Therefore, it is contemplated to cover the present invention and any and all modifications, variations, or equivalents that fall within the true spirit and scope of the basic underlying principles disclosed and claimed herein.

1. A method of forming a magnetic resonance image of a human comprising the steps of:
applying a plurality of relatively constant spatially non-selective radio frequency pulses to an imaging volume of the human;
applying a plurality of combinations of magnitude of phase-encoding gradients in slice-selective and in-plane directions to the imaging volume of the human, wherein the plurality of combinations is adapted to undersample the imaging volume in k-space;
detecting magnetic resonance imaging data from the imaging volume using a plurality of receiver coils; and
forming the magnetic resonance image of the imaging volume.

2. The method of forming a magnetic resonance image as in claim 1 wherein the step of undersampling in k-space further comprises undersampling by a factor of at least two.

3. The method of forming a magnetic resonance image as in claim 1 further comprising injecting the human with a contrast agent.

4. The method of forming a magnetic resonance image as in claim 3 further comprising periodically acquiring a subset of the magnetic resonance imaging data over a portion of the imaging volume to produce a series of time-resolved images that are different than the formed image.

5. The method of forming a magnetic resonance image as in claim 4 further comprising reducing a scan time of the time-resolved images by reducing a number of the plurality of combinations or increasing an undersampling factor of the portion of the imaging volume.

6. The method of forming a magnetic resonance image as in claim 4 further comprising interleaving the acquisition of the subset of data with the acquisition of the formed image data.

7. The method of forming a magnetic resonance image as in claim 1 further comprising sampling the imaging volume using a magnetic field strength up to 3 tesla.

8. The method of forming a magnetic resonance image as in claim 1 further comprising forming a three-dimensional image of an exterior of the human.

9. The method of forming a magnetic resonance image as in claim 8 wherein the step of forming the three-dimensional image further comprising tracing a boundary of the imaging volume in a first, second and third dimension.

10. The method of forming a magnetic resonance image as claim 8 further comprising displaying the three-dimensional image on a graphical user interface.

11. The method of forming a magnetic resonance image as in claim 8 further comprising selecting a viewing plane of the three-dimensional image using the graphical user interface so as to identify a position of additional magnetic resonance images that are subsequently acquired.

12. The method of forming a magnetic resonance image as in claim 1 further comprising defining the imaging volume as being a whole body of the human.

13. An apparatus for forming a magnetic resonance image of a human comprising the steps of:
a body coil adapted to apply a plurality of relatively constant spatially non-selective radio frequency pulses to an imaging volume of the human;
a controller adapted to apply a plurality of combinations of magnitude of phase-encoding gradients in slice-selective and in-plane directions to the imaging volume of the human, wherein the plurality of combinations is adapted to undersample the imaging volume in k-space;
a phased array have a plurality of receiver coils adapted to detect magnetic resonance imaging data from the imaging volume; and
a display processor adapted to form the magnetic resonance image of the imaging volume.

14. The apparatus for forming a magnetic resonance image as in claim 13 wherein the controller undersamples by factor of at least two.

15. The apparatus for forming a magnetic resonance image as in claim 13 further comprising a contrast agent injected into human.

16. The apparatus for forming a magnetic resonance image as in claim 15 further comprising a series of time-resolved data pulse sequences adapted to periodically acquire a subset of the magnetic resonance imaging data over a portion of the imaging volume to produce a series of time-resolved images that are different than the formed image.

17. The apparatus for forming a magnetic resonance image as in claim 16 wherein the time-resolved data pulse sequences further comprises a relatively small number of combinations of the plurality of combinations or increased undersampling factor for collecting data from the portion of the imaging volume.

18. The apparatus for forming a magnetic resonance image as in claim 16 further comprising the time-resolved data pulse sequences interleaved with high spatial resolution data pulse sequences.

19. The method of forming a magnetic resonance image as in claim 13 further comprising a magnetic field strength up to 3 tesla.

20. The method of forming a magnetic resonance image as in claim 13 wherein the formed magnetic resonance images further comprises a three-dimensional image of a surface of the volume.

21. The method of forming a magnetic resonance image as claim 20 further comprising a graphical user interface for displaying the three-dimensional image.

22. The method of forming a magnetic resonance image as in claim 21 further comprising a cursor adapted to select a viewing plane of the three-dimensional image.

23. A method of forming a magnetic resonance image of a human comprising the steps of:
applying a plurality of high spatial resolution pulse sequences to an imaging volume of the human;
applying a plurality of time-resolved pulse sequences to an imaging volume of the human, wherein the time-resolved pulse sequences are interleaved with the high spatial resolution pulse sequences, wherein the high spatial resolution pulse sequences and the time-resolved pulse sequences are different and wherein each pulse sequence of the high spatial resolution pulse sequences and the time-resolved pulse sequences includes a relatively constant spatially non-selective radio frequency pulse;
detecting magnetic resonance imaging data from the imaging volume based upon the high spatial resolution data pulse sequences and the time-resolved data pulse sequences; and
forming a magnetic resonance image of the imaging volume from one of the high spatial resolution pulse sequences and the time-resolved pulse sequences.

24. The method of forming a magnetic resonance image of a human as in claim 23 wherein the steps of applying the pulse sequences of the high spatial resolution pulse sequences and the time-resolved pulse sequences further comprises applying a plurality of combinations of magnitude of phase-encoding gradients in slice-selective and in-plane directions to the imaging volume of the human, wherein the plurality of combinations is adapted to undersample the imaging volume in k-space.

25. The method of forming a magnetic resonance image as in claim 24 wherein the step of undersampling in k-space further comprises undersampling by a factor of at least two.

26. The method of forming a magnetic resonance image as in claim 24 further comprising injecting the human with a contrast agent.

27. The method of forming a magnetic resonance image as in claim 26 further comprising periodically acquiring a subset of the magnetic resonance imaging data over a portion of the imaging volume to produce a series of time-resolved images that are different than the formed image.

28. The method of forming a magnetic resonance image as in claim 27 further comprising reducing a scan time of the time-resolved images by reducing a number of the plurality of combinations or increasing an undersampling factor of the portion of the imaging volume.

29. The method of forming a magnetic resonance image as in claim 23 further comprising sampling the imaging volume using a magnetic field strength up to 3 tesla.

30. The method of forming a magnetic resonance image as in claim 23 further comprising forming a three-dimensional image of an exterior of the human.

31. The method of forming a magnetic resonance image as claim 30 wherein the step of forming the three-dimensional image further comprising tracing a boundary of the image volume in a first, second and third dimension.

32. The method of forming a magnetic resonance image as in claim 30 further comprising displaying the three-dimensional image on a graphical user interface.

33. The method of forming a magnetic resonance image as claim 30 further comprising selecting a viewing plane of the three-dimensional image using the graphical user interface so as to identify a position of additional magnetic resonance images that are subsequently acquired.

34. The method of forming a magnetic resonance image as in claim 23 further comprising defining the imaging volume as being a whole body of the human.

35. A method of forming a magnetic resonance image of a human comprising the steps of:
applying a plurality of pulse sequences to an imaging volume of the human;
identifying a periphery of a body of the human based upon the plurality of pulse sequences;
forming a three-dimensional image on a display based upon the identified periphery.

36. The method of forming a magnetic resonance image as in claim 35 wherein the step of applying a plurality of pulse sequences further comprises applying a relatively constant spatially non-selective radio frequency pulse during each pulse sequence of the plurality of pulse sequences.

37. The method of forming a magnetic resonance image as claim 36 wherein the step of applying a plurality of pulse sequences further comprises applying a plurality of combinations of magnitude of phase-encoding gradients in slice-selective and in-plane directions to the imaging volume of the human, wherein the plurality of combinations is adapted to undersample the imaging volume in k-space.

38. The method of forming a magnetic resonance image as in claim 37 wherein the step of applying a plurality of pulse sequences further comprises detecting magnetic resonance imaging data from the imaging volume using a plurality of receiver coils.

39. The method of forming a magnetic resonance image as in claim 38 wherein the step of detecting magnetic resonance imaging data from the imaging volume using a plurality of receiver coils further comprises forming the magnetic resonance image of a slice of the imaging volume.

40. The method of forming a magnetic resonance image as claim 34 wherein the step of identifying a periphery of the body of the human further comprises automatically determining minimum and maximum phase encoding gradients in a slice-selective and also in an in-plane direction that identify voxels on opposing sides of the periphery of the body.

41. The method of forming a magnetic resonance image as in claim 40 wherein the step of automatically determining minimum and maximum phase encoding gradients further comprises automatically determining an incremental gradient step size based upon a slice thickness or a number of slices between peripheral values in the slice-selective and in-plane directions.

42. The method of forming a magnetic resonance image as in claim 41 wherein the applied plurality of pulse sequences further comprises a plurality of high spatial resolution pulse sequences interleaved with a plurality of time-resolved pulse sequences.

43. The method of forming a magnetic resonance image as in claim 42 wherein the step of forming the three-dimensional image on the display further comprises selecting a viewing slice using a graphical user interface on the display.

44. The method of forming a magnetic resonance image as in claim 43 wherein the step of selecting the slice further comprises displaying a high spatial resolution image in a viewing slice window.

45. The method of forming a magnetic resonance image as in claim 44 wherein the step of selecting the slice further comprises displaying a time-resolved image sequence in a viewing slice window.

46. The method of forming a magnetic resonance image as claim 45 wherein the step of selecting the slice further comprises displaying a time-resolved image sequence in a viewing slice window superimposed on the high spatial resolution image.
